(19) United States
(12) Patent Application Publication
(10) Pub. No.: US 2021/0395200 A1
(43) Pub. Date: Dec. 23, 2021

(54) SYNTHETIC PROCESSES FOR THE PRODUCTION OF 1-((3S,4R)-4-(2,6-DIFLUORO-4-METHOXYPHENYL)-2-OXOPYRROLIDIN-3-YL)-3-PHENYLUREA

(71) Applicant: BRISTOL-MYERS SQUIBB COMPANY, Princeton, NJ (US)

(72) Inventors: Adrian Ortiz, Oak Park, CA (US); Alina Borovika, Brooklyn, NY (US); Sergei Kolotuchin, Roselle Park, NJ (US); Michael R. Luzung, Jersey City, NJ (US); Jeffrey A. Nye, Highland Park, NJ (US); Yichen Tan, East Brunswick, NJ (US); Serge Zaretsky, Fords, NJ (US); Jason J. Zhu, East Brunswick, NJ (US); Martin D. Eastgate, Titusville, NJ (US)

(21) Appl. No.: 17/292,123

(22) PCT Filed: Nov. 15, 2019
(86) PCT No.: PCT/US2019/061650

§ 371 Date: May 7, 2021

Related U.S. Application Data
(60) Provisional application No. 62/768,266, filed on Nov. 16, 2018.

(51) Int. Cl. C07D 207/273

(52) U.S. Cl. CPC C07D 207/273

(57) ABSTRACT

Highly efficient processes are provided for preparing key intermediates in the synthesis of compounds (I). The process are broadly applicable and can provide selected components having a variety of substituents. Some intermediates are claimed.

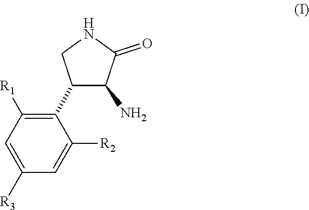

### CROSS REFERENCE TO RELATED APPLICATIONS

This application is entitled to priority pursuant to 35 U.S.C. § 119(e) to U.S. provisional patent application No. 62/768,266, filed Nov. 16, 2018, which is incorporated herein in its entirety.

### FIELD OF THE INVENTION

The invention generally relates to several improved processes for the preparation of 1-((3S,4R)-4-(2,6-difluoro-4-methoxyphenyl)-2-oxopyrrolidin-3-yl)-3-phenylurea, an FPR2 agonist useful for the treatment of heart diseases such as heart failure.

### BACKGROUND OF THE INVENTION

Heart disease is an increasingly prevalent condition that exerts a significant clinical and economic burden. The increase in prevalence is driven by patients surviving myocardial infarctions leading to cumulative myocardial damage that progressively leads to adverse cardiac remodeling and left ventricular dysfunction (Viau D M et al., Heart, 2015, 101, 1862-7., Paulus W J., Tschope C., J. Am. Coll. Cardiol., 2013, 62, 263-71). Among various heart diseases, heart failure is major health problem in the United States and elsewhere. In the United States, heart failure affects over 5 million people with approximately half a million new cases occurring each year. Heart failure is the leading cause of hospitalizations in people over 65 years in age. Despite the growing prevalence and social burden of this disease, there have been very few, if any, recent advances in treatment. Standard of care for acute coronary syndrome (ACS) patients after post-myocardial infarctions includes aspirin, statins, beta-blockers, and ACE inhibitor/ARB therapies (Zouein F A et al., J. Cardiovasc. Pharmacol., 2013, 62, 13-21). Therefore, there is unmet medical need to develop pharmaceutical agents that specifically target heart failure.

Recently, FPR2 agonists useful in the treatment of immunological diseases have been reported. One such class of compounds is substituted phenylureas described in U.S. Pat. No. 9,822,069, which is hereby incorporated by reference in its entirety. For example, Compound 1 of the following structure:

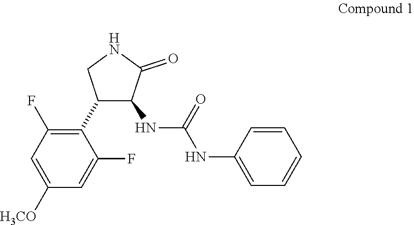

has been shown to possess robust FPR2 agonist activity. The patent discloses a multistep synthesis process for preparing the compound. However, there are difficulties associated with the adaptation of the multistep synthesis disclosed in the patent to a larger scale synthesis, such as production in a pilot plant or on a manufacturing scale. Desired is a process that is suitable for preparing larger quantities of Compound 1 than is typically prepared by laboratory scale processes. Also desired is a process that could minimize or eliminate the number of genotoxic impurities and provide higher yields of Compound 1 than the previously disclosed processes.

The present invention is directed to one or both of these as well as other important embodiments.

### SUMMARY OF THE INVENTION

Provided herein are processes for the production of key intermediates in the preparation of Compound 1, namely phenylurea 1-((3S,4R)-4-(2,6-difluoro-4-methoxyphenyl)-2-oxopyrrolidin-3-yl)-3-phenylurea, that are cost effective and readily scaleable with commercial reagents. Surprisingly and without wishing to be bound by theory, the key intermediates generated by these processes have been found to be stable and non-toxic.

In one embodiment, the present invention provides a process for the preparation of a compound of Formula (I)

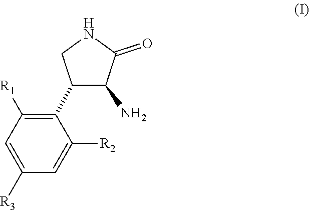

or a salt thereof, wherein each of R₁ and R₂ is halogen and R₃ is C₁₋₄ alkoxy, comprising the steps of

(1) condensing a sulfonamide chiral auxiliary with a substituted phenyl aldehyde in a solvent to provide an imine product;

(2) reacting the resulted imine product with a sulfonium-ylide to afford an aziridine electrophile;

(3) reacting the aziridine electrophile with an enolate nucleophile to afford the compound of Formula (I).

In another embodiment, the present invention provides a process for the preparation of Compound (XIV):

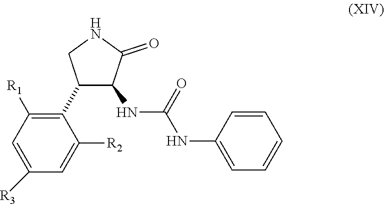

wherein each of R₁ and R₂ is halogen and R₃ is C₁₋₄ alkoxy, comprising the steps of

(1) condensing a sulfonamide chiral auxiliary with a substituted phenyl aldehyde in a solvent to provide an imine product;

(2) reacting the resulted imine product with a sulfonium-ylide to afford an aziridine electrophile;

(3) reacting the aziridine electrophile with an enolate nucleophile to afford the compound of Formula (I);

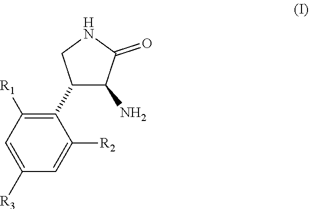

wherein R₁, R₂, and R₃ are as defined above;

(4) coupling the compound of Formula (I) with a phenylisocyanate in the presence of an alcoholic solvent and a base to afford the compound of Formula (XIV).

In another embodiment, the present invention provides a process for the preparation of Compound 1, comprising the steps of

(1) condensing a sulfonamide chiral auxiliary with Compound 2 in a solvent to provide Compound 3;

(2) reacting Compound 3 with a sulfonium-ylide to afford Compound 4;

(3) reacting Compound 4 with Compound 5 to afford Compound 8;

(4) coupling Compound 8 with a phenylisocyanate in the presence of an alcoholic solvent and a base to afford Compound 1.

In one embodiment of the above mentioned process, the phenyl aldehyde is a compound of Formula (II):

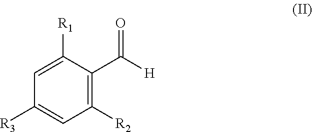

wherein each of R₁ and R₂ is halogen and R₃ is C₁₋₄ alkoxy.

In another embodiment of the above mentioned process, the sulfonamide chiral auxiliary is

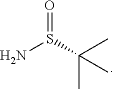

In another embodiment of the above mentioned process, the imine product is a compound of Formula (III):

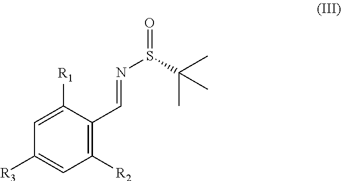

wherein each of R₁ and R₂ is halogen and R₃ is C₁₋₄ alkoxy.

In another embodiment of the above mentioned process, the sulfonium-ylide is generated from a suitable salt and a suitable base.

In another embodiment of the above mentioned process, the aziridine electrophile is a compound of Formula (IV):

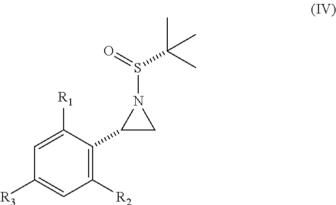

wherein each of R₁ and R₂ is halogen and R₃ is C₁₋₄ alkoxy.

In another embodiment of the above mentioned process, the enolate nucleophile is a glycine imine derivative of Formula (V):

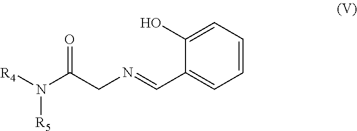

wherein

R₄ and R₅ are independently selected from the group consisting of H, C₁₋₃ alkyl, C₃₋₆ cycloalkyl, phenyl, and 5- to 6-membered heterocycle containing carbon atoms and 1-4 heteroatoms selected from the group consisting of N, O, and S.

In another embodiment of the above mentioned process, the compound of Formula (V) is reacted with a base in an organic solvent in the presence of LiCl to form a lithium dianion.

In another embodiment of the above mentioned process, process Step (3) further comprises the steps of

3(a) replacing the sulfonamide auxiliary protecting group of the compound of Formula (VI) with a Schiff base protecting group; and

3(b) removing the Schiff base protecting group and cyclizing the compound.

In another embodiment of the above mentioned process, an intermediate generated from Step 3(a) is a compound of Formula (VI):

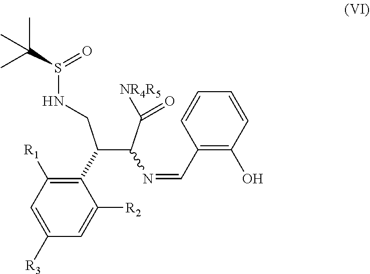

wherein:

each of R₁ and R₂ is halogen;

R₃ is C₁₋₄ alkoxy; and

R₄ and R₅ are independently selected from the group consisting of H and C₁₋₃ alkyl.

In another embodiment of the above mentioned Step 3(b), the compound of Formula (VI) is reacted with an acid and in the presence of 2-hydroxybenzaldehyle to afford a compound of Formula (VII):

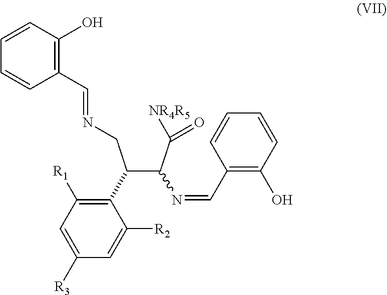

wherein:

each of R₁ and R₂ is halogen;

R₃ is C₁₋₄ alkoxy; and

R₄ and R₅ are independently selected from the group consisting of H and C₁₋₃ alkyl.

In another embodiment of the above mentioned process, the compound of Formula (VII) is treated with a chiral acid in a mixture of water and an alcohol to afford a compound of Formula (I).

In another embodiment, the present invention provides an alternative process for the preparation of a compound of Formula (I):

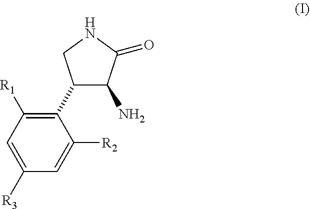

wherein each of R₁ and R₂ is halogen and R₃ is C₁₋₄ alkoxy, comprising the steps of

(1) reacting the compound of Formula (IV):

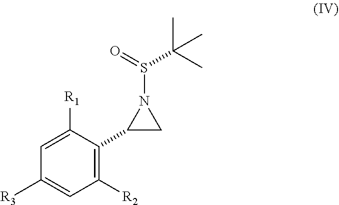

with a benzophenone glycine imine ester;

(2) treating the resultant product with a chiral acid in an alcohol to afford a compound of Formula (I).

In another embodiment, the present invention provides an alternative process for the preparation of a compound of Formula (I):

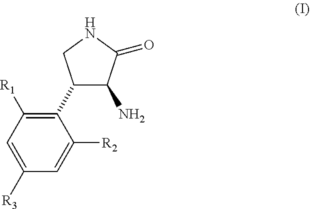

wherein each of R₁ and R₂ is halogen and R₃ is C₁₋₄ alkoxy, comprising the steps of

(1) reacting the compound of Formula (IV):

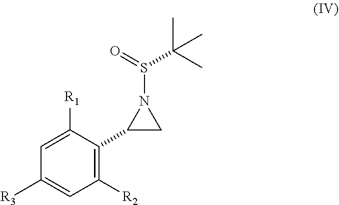

with a malonate derivative;

(2) treating the resultant product with a base to afford a compound of Formula (VIII):

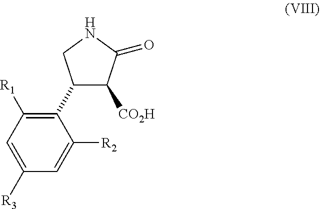

(3) convening the compound of Formula (VIII) into a hydroxamic acid of Formula (IX):

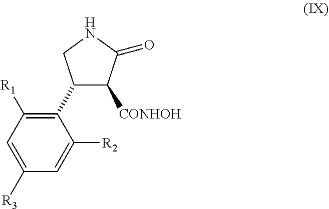

(4) Converting the hydroxoamic acid by Lossen Rearrangement to afford a compound of Formula (X):

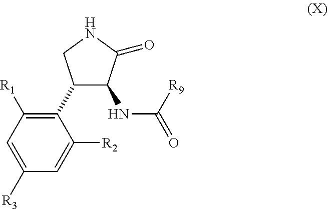

wherein R₉ is 5- to 6-membered heterocycle containing carbon atoms and 1-4 heteroatoms selected from the group consisting of N, O, and S;

(5) treating the resultant product with tartaric acid to afford a compound of Formula (I).

In another embodiment, the present invention provides an alternative process for the preparation of a compound of Formula (I):

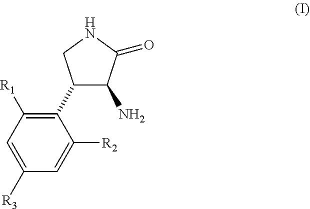

wherein each of R₁ and R₂ is halogen and R₃ is C₁₋₄ alkoxy, comprising the steps of

(1) oxidizing the compound of Formula (IV):

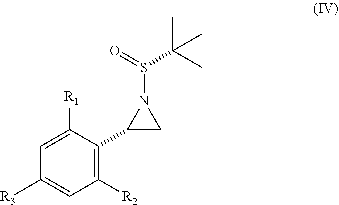

with an oxidizing agent to afford a compound of Formula (XI):

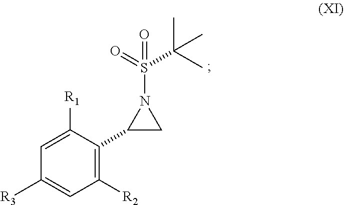

(2) reacting the compound of Formula (XI) with a glycine imine ester; and

(3) treating the resultant product with a chiral acid in an alcohol to afford a compound of Formula (I).

In another embodiment, the present invention provides an alternative process for the preparation of a compound of Formula (I):

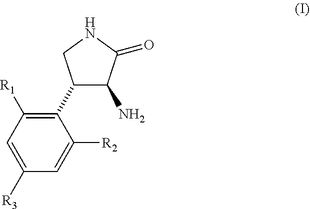

wherein each of R₁ and R₂ is halogen and R₃ is C₁₋₄ alkoxy, comprising the steps of

(1) reacting the compound of Formula (XI):

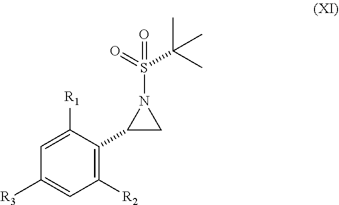

with a substituted acetamide and cyclizing the compound to afford a compound of Formula (XII):

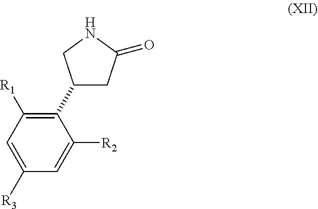

(2) aminating the compound of Formula (XII) with DBAD to afford a compound of Formula (XIII):

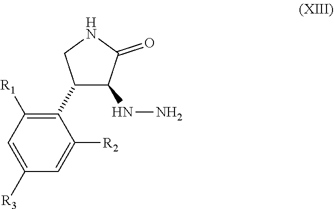

(3) reducing the compound of Formula (XIII) to afford a compound of Formula (I).

In another embodiment, the present invention provides methods for treating a thromboembolic disorder, comprising administering to a mammalian species, preferably a human, in need thereof, a therapeutically effective amount of Compound 1, wherein Compound 1 is prepared utilizing the novel process steps of the invention.

### BRIEF DESCRIPTION OF THE DRAWING

FIG. 1 is a general synthetic scheme to make Compound 1. As demonstrated in FIG. 1, the C₃-C₄ bond and 5-membered pyrrolidone are forged through a formal [3+2] transformation. If a benzylic electrophile enantioenriched at C4 (A) is utilized with a glycine enolate equivalent nucleophile (B), the formation of the pyrrolidone can be accomplished without initially being required to control the stereochemistry at C3. Correction of this C3 stereocenter is then carried out through a dynamic resolution process to afford the thermodynamically favored trans configuration (C3-C4).

### DETAILED DESCRIPTION OF THE INVENTION

### Definitions

Listed below are definitions of various terms used to describe the present invention. These definitions apply to the terms as they are used throughout the specification (unless they are otherwise limited in specific instances) either individually or as part of a larger group.

Throughout the specification, groups and substituents thereof may be chosen by one skilled in the field to provide stable moieties and compounds.

As used herein, “a” or “an” means one or more unless otherwise specified.

As used herein, “about” refers to any values, including both integers and fractional components that are within a variation of up to ±10% of the value modified by the term “about.”

As used herein, “include,” “including,” “contain,” “containing,” “has,” or “having,” and the like, mean “comprising.”

As used herein, the term “alkyl” refers to a straight or branched, saturated aliphatic radical containing one to ten carbon atoms, unless otherwise indicated e.g., alkyl includes methyl, ethyl, propyl, isopropyl, butyl, sec-butyl, isobutyl, tert-butyl, and the like. The term “lower alkyl” refers to an alkyl radical having from one to four carbon atoms.

The term “alkoxy” refers to a group having the formula —O-alkyl, in which an alkyl group, as defined above, is attached to the parent molecule via an oxygen atom. The alkyl portion of an alkoxy group can 1 to 10 carbon atoms (i.e., alkoxy), or 1 to 6 carbon atoms (i.e., C₁-C₁₀ alkoxy). Examples of suitable alkoxy groups include, but are not limited to, methoxy (—O—CH₃ or —OMe), ethoxy (—OCH₂CH₃ or —OEt), t-butoxy (—O—C(CH₃)₃ or —OtBu) and the like.

The term “aryl” as used herein, refers to a group of atoms derived from a molecule containing aromatic ring(s) by removing one hydrogen that is bonded to the aromatic ring(s). Heteroaryl groups that have two or more rings must include only aromatic rings. Representative examples of aryl groups include, but are not limited to, phenyl and naphthyl. The aryl ring may be unsubstituted or may contain one or more substituents as valence allows. Exemplary substituents include F, Cl, Br, I, —OH, C₁₋₆ alkyl, C₁₋₄ fluoroalkyl, —NO₂, —NH₂, and —O(C₁₋₃ alkyl).

The term “substituted phenyl” refers to an additional substituent group selected from halogen (preferably fluoro, chloro, or bromo), hydroxy, amino, mercapto, and the like on the phenyl ring.

The term “reducing agent” refers to any reagent that will decrease the oxidation state of a carbon atom in the starting material by either adding a hydrogen atom to this carbon or adding an electron to this carbon and as such would be obvious to one of ordinary skill and knowledge in the art. Examples include, but are not limited to, borane-dimethyl sulfide complex, 9-borabicyclo[3.3.1]nonane (9-BBN), catechol borane, lithium borohydride, sodium borohydride, sodium borohydride-methanol complex, potassium borohydride, sodium hydroxyborohydride, lithium triethylborohydride, lithium n-butylborohydride, sodium cyanoborohydride, calcium (II) borohydride, lithium aluminum hydride, diisobutylaluminum hydride, n-butyl-diisobutylaluminum hydride, sodium bis-methoxyethoxyaluminum hydride, triethoxysilane, diethoxymethylsilane, lithium hydride, lithium, sodium, hydrogen Ni/B, and the like. Certain acidic and Lewis acidic reagents enhance the activity of reducing reagents. Examples of such acidic reagents include: acetic acid, methanesulfonic acid, hydrochloric acid, and the like. Examples of such Lewis acidic reagents include: trimethoxyborane, triethoxyborane, aluminum trichloride, lithium chloride, vanadium trichloride, dicyclopentadienyl titanium dichloride, cesium fluoride, potassium fluoride, zinc (II) chloride, zinc (II) bromide, zinc (II) iodide, and the like.

The term “removable protecting group” or “protecting group” refers to any group which when bound to a functionality, such as the oxygen atom of a hydroxyl or carboxyl group or the nitrogen atom of an amine group, prevents reactions from occurring at these functional groups and which protecting group can be removed by conventional chemical or enzymatic steps to reestablish the functional group. The particular removable protecting group employed is not critical.

“Stable compound” and “stable structure” are meant to indicate a compound that is sufficiently robust to survive isolation to a useful degree of purity from a reaction mixture, and formulation into an efficacious therapeutic agent. The present invention is intended to embody stable compounds.

The compounds of the present invention are intended to include all isotopes of atoms occurring in the present compounds. Isotopes include those atoms having the same atomic number but different mass numbers. By way of general example and without limitation, isotopes of hydrogen include deuterium (D) and tritium (T). Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. For example, methyl (—CH₃) also includes deuterated methyl groups such as —CD₃.

### ABBREVIATIONS

AcOH acetic acid
anhyd. anhydrous
aq. aqueous
Bn benzyl
Boc tert-butoxycarbonyl
bus tert-butylsulfonyl
CDI Carbonyldiimidazole
DBAD Di-tert-butyl azodiacarboxylate
DKR Dynamic kinetic resolution
DMAc N,N-dimethyl acetamide
DMAP 4-dimethylaminopyridine
DMF dimethylformamide
DMSO dimethylsulfoxide
DPPOH diphenyl phosphate
Et ethyl
Et₃N triethyl amine
EtOH ethanol
H or H₂ hydrogen
h, hr or hrs hour(s)
IPA isopropyl alcohol
i-Pr isopropyl
HPLC high pressure liquid chromatography
IPAc isopropyl acetate
LC liquid chromatography
LCMS liquid chromatography mass spectroscopy
LiHMDS Lithium hexamethyldisilizane
M moles/liter
m-CPBA meta-Chloroperoxybenzoic acid
mM millimoles/liter
Me methyl
MeOH methanol
MeTHF methyl tetrahydrofuran
MHz megahertz
min. minute(s)
mins minute(s)
MS mass spectrometry
MSA methanesulfonic acid
MTBE methyl tetrabutyl ether
NaHMDS Sodium hexamethyldisilizane
NaOMe sodium methoxide
nM nanomolar
Ph phenyl
Ret Time or Rt retention time
sat. saturated
SFC supercritical fluid chromatography
TBD 1,3,4,6,7,8-hexahydro-2H-pyrimido[1,2-a]pyrimidine
t-BuOK Potassium tert-butoxide
t-BuOH tertiary butanol
TBTU O-(Benzotriazol-1-yl)-N,N,N′,N′-tetramethyluronium tetrafluoroborate
THF tetrahydrofuran
TMSCl trimethyl silyl chloride

### EMBODIMENTS OF THE INVENTION

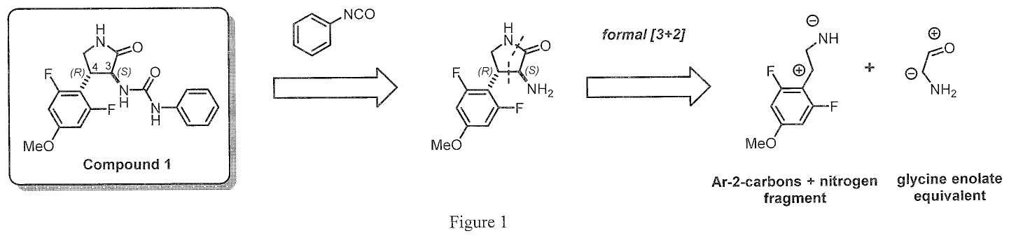

The present invention resides in a number of synthetic intermediates and processes for preparing those intermediates and Compound 1. The processes illustrated in FIG. 1 are able to minimize or eliminate the number of genotoxic impurities (GTI's) in the synthetic route, complete the synthesis in fewer than seven steps as compared to the process described in U.S. Pat. No. 9,822,069.

General aspects of these exemplary methods are described in the schemes and the Examples. Each of the products of the following processes is optionally separated, isolated, and/or purified prior to its use in subsequent processes.

Generally, the reaction conditions such as temperature, reaction time, solvents, work-up procedures, and the like, will be those common in the art for the particular reaction to be performed. Typically the temperatures will be −100° C. to 200° C., solvents will be aprotic or protic, and reaction times will be 10 seconds to 10 days. Work-up typically consists of quenching any unreacted reagents followed by partition between a water/organic layer system (extraction) and separating the layer containing the product.

Oxidation and reduction reactions are typically carried out at temperatures near room temperature (about 20° C.), although for metal hydride reductions frequently the temperature is reduced to 0° C. to −100° C., solvents are typically aprotic for reductions and may be either protic or aprotic for oxidations. Reaction times are adjusted to achieve desired conversions.

Scheme 1 provides more detailed descriptions of the reaction sequences. Each step of the preparation method will now be described in more detail.

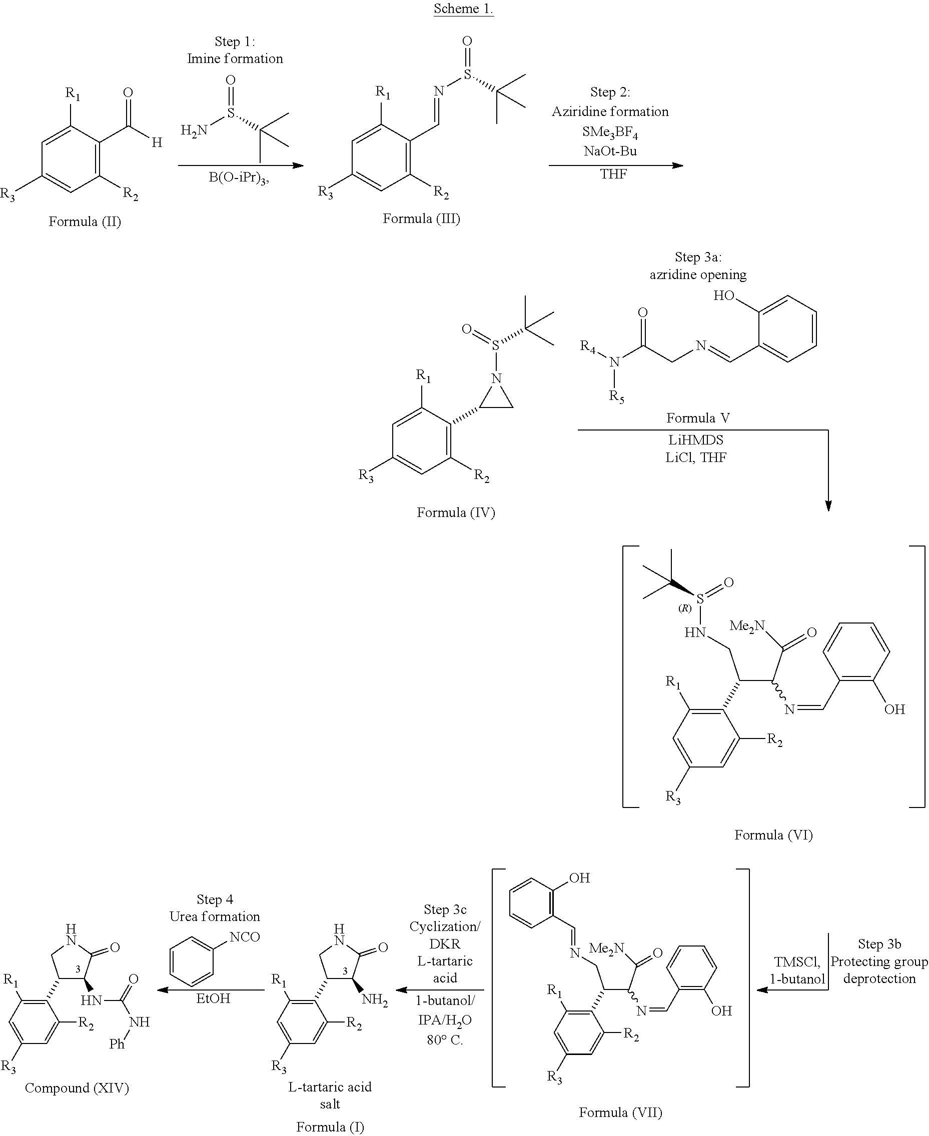

### Step 1 Imine Formation

In this condensation reaction, an Ellman sulfonamide chiral auxiliary is reacted with a substituted phenyl aldehyde of the compound of Formula (II):

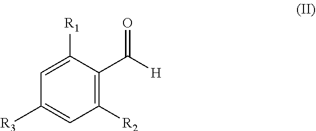

wherein each of R₁ and R₂ is halogen and R₃ is C₁₋₄ alkoxy to afford a compound of Formula (III):

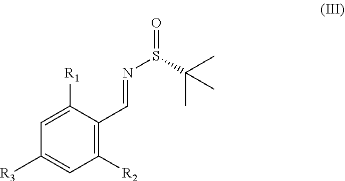

This transformation was mediated by heat and in the presence of B(i-PrO)₃.

The Ellman sulfonamide chiral auxiliary may be selected from

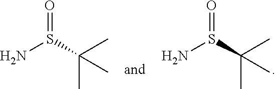

This transformation is mediated by heating and by the use dehydrating reagents, which also serve as solvents for the reaction. Different combinations of solvents and dehydrating reagents such as Ti(OEt)₄ and B(i-PrO)₃ may be used. Although, Soluble Ti(OEt)₄ is the most common dehydrating reagent, it requires extensive processing, i.e., aqueous work up and/or additional filtration to remove titanium salts, and insoluble inorganics such Na₂SO₄ or CuSO₄. The preferred reagent for this reaction is B(i-PrO)₃. Upon cooling the reaction, the compound of Formula (III) can be crystallized and filtered directly without additional processing. Typical isolated yields range between 80-90%.

### Step 2 Aziridine Formation

In this step, the compound Formula (III) is reacted with a sulfonium-ylide generated from a salt and a base in a solvent at a temperature in the range of about −10 to 20° C. to generate a compound of Formula (IV) in yields ranging between 50-60%.

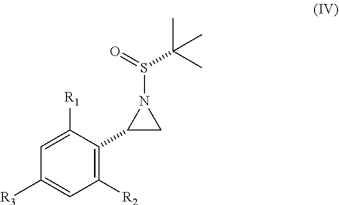

In the transformation, diastereoselectivity is critical in ensuring enantioenrichment of the final target. Therefore, salts and bases that can enhance the diastereoselectivity should be used. Suitable salts include, but are not limited to, SMe₃BF₄, SMe₃Cl, SMe₃Br, SMe₃I, and SMe₃PF₆. Among these, SMe₃BF₄ is preferred because of its enhanced solubility and high diastereoselectivity, which can be as high as 90:10.

Suitable bases are hydroxides, with Li⁺, Na⁺, K⁺, Cs⁺, NH₄⁺ as counter cation. Examples are sodium hydroxide, potassium hydroxide, potassium t-butoxide, sodium t-butoxide, sodium methoxide, potassium methoxide, sodium ethoxide, potassium ethoxide, sodium tert-pentoxide (NaOt-Amyl), potassium tert-pentoxide sodium isopropoxide, and potassium isopropoxide. Among them, NaOt-Amyl possess the ideal base strength, i.e., strong enough to deprotonate the SMe₃BF₄ and generate the necessary ylide, yet weak enough that the generated aziridine product does not decompose in its presence. The properties NaOt-Amyl enable an addition order in which NaOt-Amyl is added last and ylide is formed and consumed rapidly. This type of operation is important to ensure robustness. If the ylide is formed in the absence of compound of Formula (III), it will react with itself/polymerize over time.

Examples of suitable solvents include, but are not limited to, polar aprotic solvents such as dimethyl formamide, dimethyl sulfoxide, and N-methylpyrrolidinone; etheral solvents such tetrahydrofuran (THF), 2-methyl tetrahydrofuran (2-MeTHF), methyl t-butyl ether (MTBE), diethoxymethane, and cyclopropylmethyl ether (CPME); hydrocarbons such as benzene, toluene, hexanes, and heptane; halogenated solvents such as dichloromethane and 1,2-dichloroethane; acetates such as ethyl acetate, isopropyl acetate, and butyl acetate, and other solvents such as acetonitrile, methyl vinyl ketone, N,N-dimethylacetamide; polar aprotic solvent such as and mixtures thereof. Preferred solvents include etheral solvents such as THF, 2-MeTHF, and diethoxymethane. In this reaction, the combination of NaOt-Amyl, SMe₃BF₄, and THF is preferred.

### Step 3: Aziridine Opening/Ring Closure

Step 3 is a 3-step telescope consisting of (3a) a C—C bond formation through an aziridine ring opening reaction, (3b) selective deprotection of the Ellman protecting group, and (3c) an intramolecular cyclization followed by salt formation to produce the compound of Formula (I) as a tartaric acid salt.

### Step 3a:

The starting materials for this step are a compound of Formula (IV) and a compound of Formula (V), wherein R₄ and R₅ are independently selected from the group consisting of H, C₁₋₃ alkyl, C₃₋₆ cycloalkyl, phenyl, and 5- to 6-membered heterocycle to obtain the compound of Formula (VI). The compound of Formula (V) can be generated according to

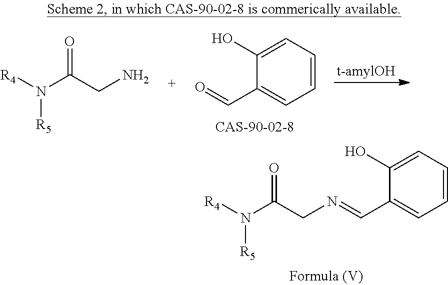

In the reaction, the Lithium dianion of the compound of Formula (V) is prepared in a solvent using a base and in the presence of LiCl. LiCl is required to increase the solubility of both mono- and dianion species and also offers the benefit of increasing the reaction kinetics for aziridine ring opening. In the absence of LiCl, the reaction is extremely heterogeneous and not able to be stirred. The base may be a strong lithiated base such as an alkyl lithiated base or aryl lithiated base. Non-limiting examples of the alkyl and aryl lithiated bases are methyl lithium, n-butyl lithium, sec-butyl lithium, tert-butyl lithium, phenyl lithium, LDA (lithium diisopropylamide), LHMDS (lithium hexamethyl disilazide), and LTMP (lithium tetramethylpiperidide).

After the dianion was formed, the compound of Formula (IV) is added and the reaction is aged at ambient temperature until reaction completion. The reaction typically requires 16 h to reach completion and generates a diatereomeric mixture of C—C bond products. The reaction is quenched and the THF is swapped to 1-butanol for the next step. The reaction temperature may be varied over a relatively wide range. The reaction is generally carried out at temperatures from 0° C. to 80° C. Preferably, the reaction is carried out from about 20° C. to about 65° C.

### Step 3b:

In this second telescoped transformation, the Ellman auxiliary of Formula (VI) is selectively removed in the presence of the Schiff base by an acid. The acid can be HCl and it can be generated in-situ by reacting silylchloride with a solvent or by addition of anhydrous HCl. This selective deprotection is quite challenging given the Schiff base is actually more acid sensitive than the Ellman group. However, under very specific conditions, this transformation can be achieved by the addition of 2-hydroxy benzaldehyde to the reaction and by maintaining strictly anhydrous conditions throughout the process, including an anhydrous neutralization of HCl with an organic base. The resulting double Schiff base product is a mixture of cis/trans diastereomers of Formula (VII). Alcoholic solvents perform best in the reaction, but 1-butanol is preferred. Other anhydrous HCl sources could be employed but TMSCl is preferred. Many organic bases could be employed for the neutralization of the HCl, but Et₃N is preferred.

### Step 3c:

In this step, the compound of Formula (VII) is treated with L-tartaric acid in a mixture of water and alcohol to afford a compound of Formula (I). MeOH, EtOH, 1-propanol, 2-propanol, 1-butanol all perform well, but a mixture of IPA/1-butanol and water is preferred. Other chiral acids can be employed, but L-tartaric acid is preferred. The compound of Formula (I) is isolated by cooling the heated reaction mixture. Typical yields over the three-step telescope range between 55-70% and the resultant L-tartaric acid salt of the compound of Formula (I) is of very high quality and purity. Without wishing to be bound by theory, the use of L-tartaric acid enables removal of any enantiomers or diastereomers of Compound I that may be present and therefor acts as a critical quality gate keeper in this process. The reaction may be carried out from about 40° C. to about 90° C. Preferably, the reaction is carried out from about 70° C. to about 85° C.

### Step 4: Urea Formation

The final step consists of the reaction of the compound of Formula (I) with phenylisocyanate in a solvent to generate Compound (1). Preferred solvent is an alcoholic solvent such as a C₁₋₆ alcoholic solvent: methanol, ethanol, propanol butanol, pentanol, and hexanol. Preferably, it is ethanol. A base such as imidazole is also used. Typical yields for this transformation range between 90-95% yield.

In the process of preparing the intermediates above, additional steps can be employed among Steps 1-4. In addition, different synthesis processes may be employed to prepare key intermediates in Scheme 1. Schemes 2-5 below show different synthesis routes of opening the aziridine ring in the process of preparing the compound of Formula (I).

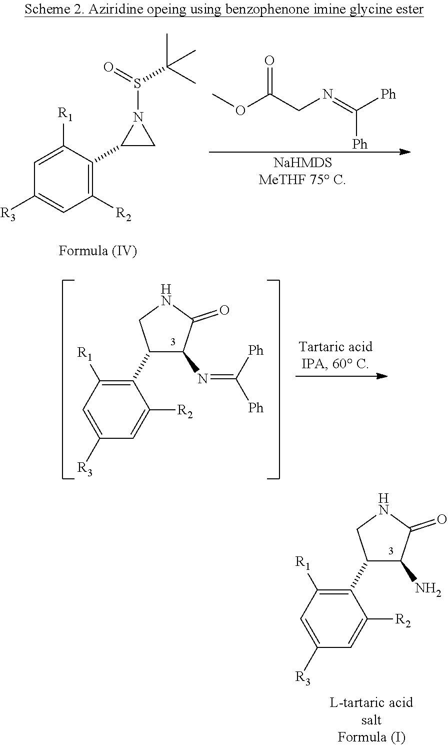

In this scheme, the Ellman aziridine of Formula (IV) is reacted with commercially available benzophenone glycine imine ethyl ester, and then proceed with the same L-tartaric acid salt formation as describe above. Typical isolated yields are about 34%.

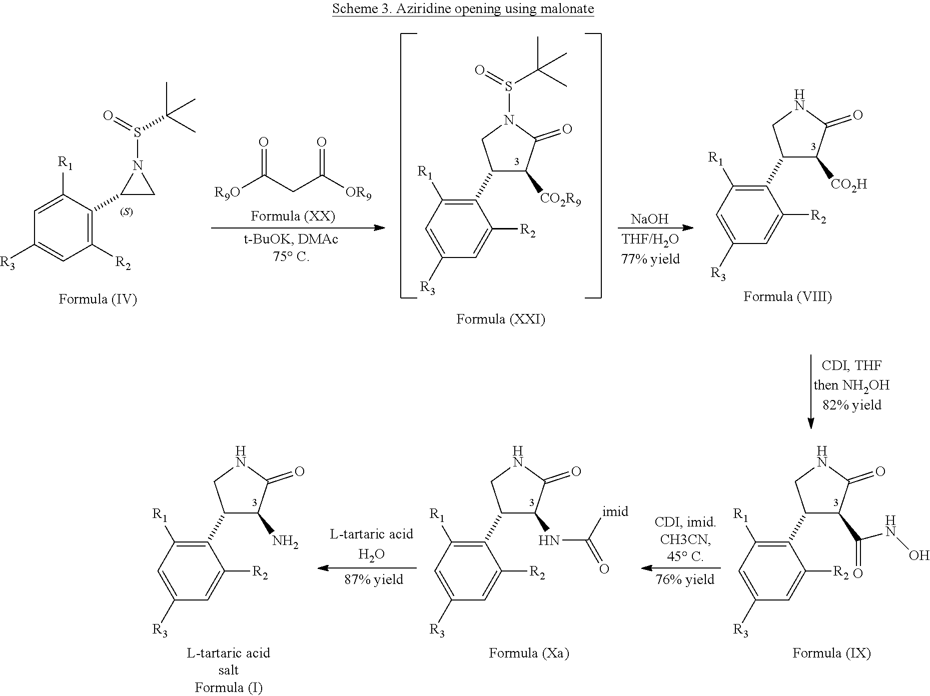

In this reaction, the compound of Formula (IV) is reacted with a compound of Formula (XX) wherein R₉ is C₁₋₃ alkyl to give rise to the compound of Formula (XXI). After treatment with a base, the resulting compound of Formula (VIII) could be isolated in 77% yield. Suitable bases are alkoxide bases such as methoxide, ethoxide, tert-butoxide, amylate, tert-amylate, with counter cations such as Li⁺, Na⁺, and K⁺ are also suitable. Preferably, the base is NaOH. The compound of Formula (IX) is then subjected to Curtius reaction or Lossen rearrangement. In both cases, the reactions converge on the imidazole adduct of Formula (X). Formula (I) could be accessed by treatment with tartaric acid and water with about 87% yield.

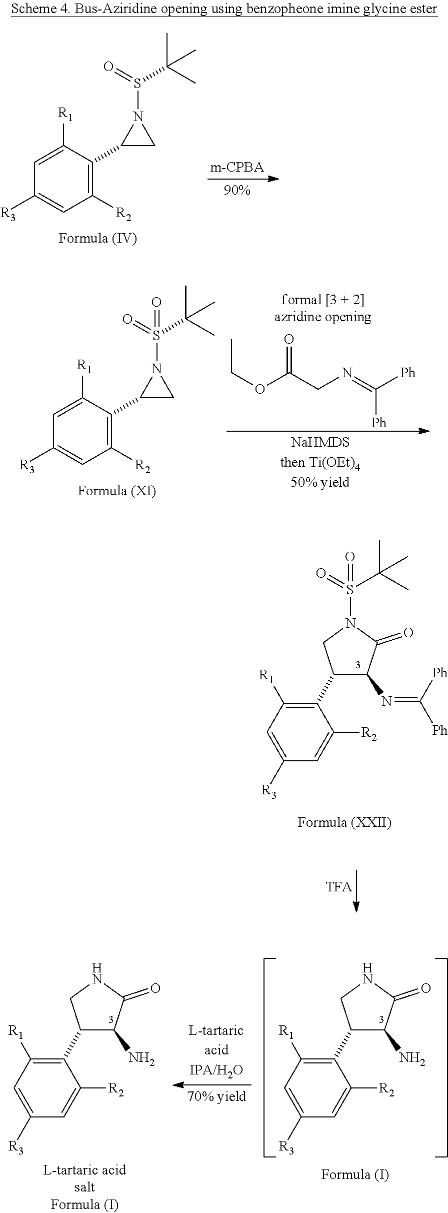

The Ellman Aziridine of Compound (IV) is reacted with an oxidizing agent to form an activated species, the Bus-aziridine of Formula (XI). The Bus-aziridine is reacted with benzophenone glycine imine ethyl ester to obtain a compound of Formula (XXII). Removal of the Bus-group was conducted with anhydrous TFA and then telescoped into the tartaric acid salt formation (70% yield).

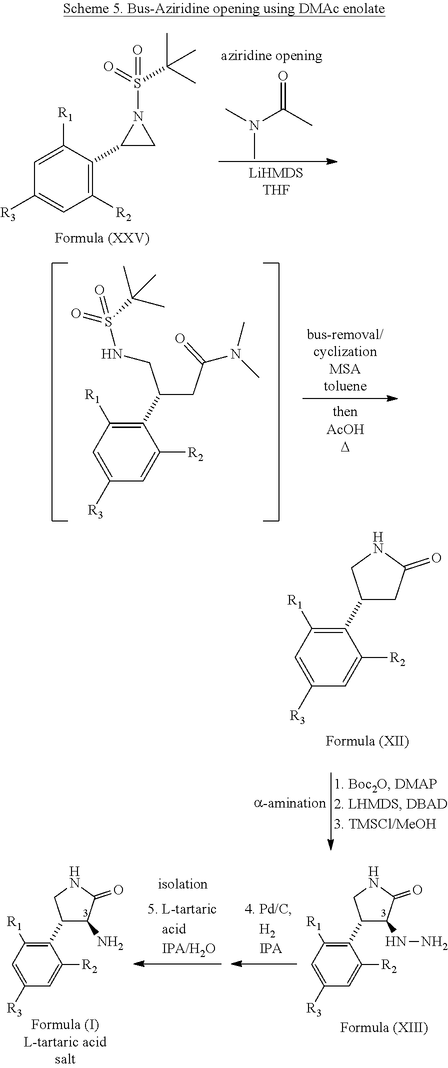

The aziridine of the compound of Formula (XI) can also be opened with another stable nucleophile, DMAc enolate. The Bus group is removed by MSA/toluene and the compound undergoes cyclization by treating it with AcOH at reflux to afford the compound of Formula (XII). Installation of the C-3 amino group can be carried out in a three-step process starting with N-Boc protection, alpha amination with DBAD, and then treatment with TMSCl, producing the resulting C-3 hydrazine intermediate of Formula (XIII).

Compound of Formula (XIII) is then subjected to a reduction step using a metal catalyst, such as Pd, Pt, Rh in the presence of hydrogen gas or a hydrogen transfer reagent such as ammonium or sodium formate in an etheral solvent or alcohol solvent to form an intermediate, which is then treated with L, tartaric acid to obtain the compound of Formula (I).

In another embodiment, the present invention provides a compound of Formula (XV):

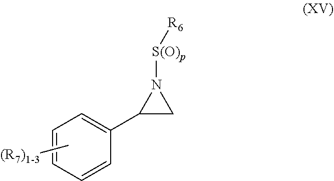

wherein

R₆ is C₁₋₆alkyl;

R₇ is selected from the group consisting of halogen, OH, C₁₋₄alkyl, C₂₋₄ alkenyl, C₁₋₄alkoxy, C₁₋₄alkylthio, C₁₋₄haloalkyl, —CH₂OH, —OCH₂F, —OCHF₂, —OCF₃, CN, —NH₂, —NH(C₁₋₄ alkyl), —N(C₁₋₄ alkyl)₂, —CO₂H, —CH₂CO₂H, —CO₂(C₁₋₄ alkyl), —CO(C₁₋₄ alkyl), —CH₂NH₂, —CONH₂, —CONH(C₁₋₄ alkyl), and —CON(C₁₋₄ alkyl)₂; and

p is an integer of 1 or 2.

In another embodiment, the present invention provides a compound having the structure:

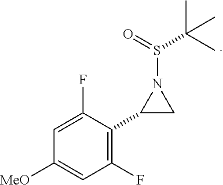

In another embodiment, the present invention provides a compound having the structure.

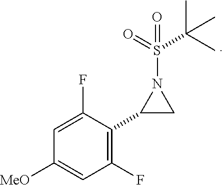

In another embodiment, the present invention provides a compound of Formula (V):

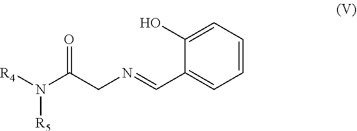

wherein R₄ and R₅ are independently selected from the group consisting of H, C₁₋₄alkyl, C₃₋₆ cycloalkyl, phenyl, and 5- to 6-membered heterocycle containing carbon atoms and 1-4 heteroatoms selected from the group consisting of N, O, and S.

In another embodiment, the present invention provides a compound having the structure.

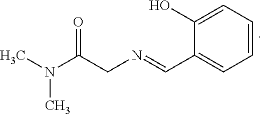

In another embodiment, the present invention provides a compound of Formula (XVII):

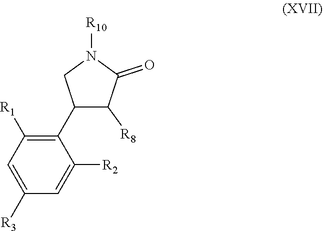

wherein

each of R₁ and R₂ is halogen;

R₃ is C₁₋₄ alkoxy;

R₈ is selected from the group consisting of —CO₂R₉, —CONH—OH, —NHCOR₉, —N═C(R₉)₂, —N(R₉)₂, —NH—NH₂; and

R₉ is selected from the group consisting of H, C₃₋₆ cycloalkyl, aryl, and 5- to 6-membered heterocycle containing carbon atoms and 1-4 heteroatoms selected from the group consisting of N, O, and S; and

R₁₀ is selected from the group consisting of H, S(O)C₁₋₆ alkyl, and S(O)₂C₁₋₆alkyl.

In another embodiment, the present invention provides a compound of Formula (XVII), wherein

each of R₁ and R₂ is F;

R₃ is methoxy;

R₈ is selected from the group consisting of —CO₂H, —CONH—OH, —NHCO-imidazole, —N═C(Ph)₂, and —NH—NH₂; and

R₁₀ is H.

In another embodiment, the present invention provides a compound of Formula (XVII), wherein

each of R₁ and R₂ is F;

R₃ is methoxy;

R₈ is selected from the group consisting of —CO₂H—CONH—OH, —NHCO-imidazole, —N═C(Ph)₂, and —NH—NH₂; and

R₁₀ is selected from the group consisting of S(O)C₁₋₆ alkyl and S(O)₂C₁₋₆alkyl.

In another embodiment, the present invention provides a compound of Formula (XVIII):

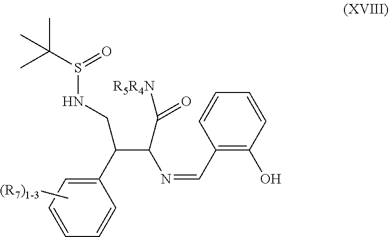

wherein R₇ is selected from the group consisting of halogen, OH, C₁₋₄alkyl, C₂₋₄ alkenyl, C₁₋₄alkoxy, C₁₋₄alkylthio, C₁₋₄haloalkyl, —CH₂OH, —OCH₂F, —OCHF₂, —OCF₃, CN, —NH₂, —NH(C₁₋₄ alkyl), —N(C₁₋₄ alkyl)₂, —CO₂H, —CH₂CO₂H, —CO₂(C₁₋₄ alkyl), —CO(C₁₋₄ alkyl), —CH₂NH₂, —CONH₂, —CONH(C₁₋₄ alkyl), and —CON(C₁₋₄ alkyl)₂; and

R₄ and R₅ are independently selected from the group consisting of H and C₁₋₃ alkyl.

In another embodiment, the present invention provides a compound of Formula (XIX):

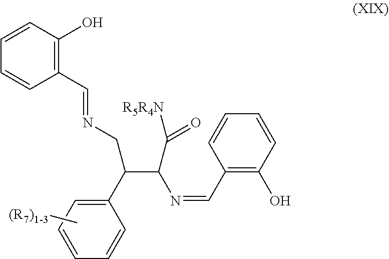

wherein R₇ is selected from the group consisting of halogen, OH, C₁₋₄alkyl, C₂₋₄ alkenyl, C₁₋₄alkoxy, C₁₋₄alkylthio, C₁₋₄haloalkyl, —CH₂OH, —OCH₂F, —OCHF₂, —OCF₃, CN, —NH₂, —NH(C₁₋₄ alkyl), —N(C₁₋₄ alkyl)₂, —CO₂H, —CH₂CO₂H, —CO₂(C₁₋₄ alkyl), —CO(C₁₋₄ alkyl), —CH₂NH₂, —CONH₂, —CONH(C₁₋₄ alkyl), and —CON(C₁₋₄ alkyl)₂; and

R₄ and R₅ are independently selected from the group consisting of H and C₁₋₃ alkyl.

### EXAMPLES

With the aim to better illustrate the present invention, the following examples are given. All reactions were performed under a nitrogen atmosphere using anhydrous techniques unless otherwise noted. Reagents were used as received from the vendors, unless otherwise noted. Quoted yields are for isolated material, and have not been corrected for moisture content. Reactions were monitored by normal or reverse phase HPLC. From the above discussion and the Example, one skilled in the art can ascertain the essential characteristics of the invention, and without departing from the spirit and scope thereof, can make various changes and modifications to adapt the invention to various uses and conditions. As a result, the invention is not limited by the illustrative examples set forth herein below, but rather is defined by the claims appended hereto.

The preparation of intermediates Compounds 1-8 are described in Scheme 6 and Examples 1-4.

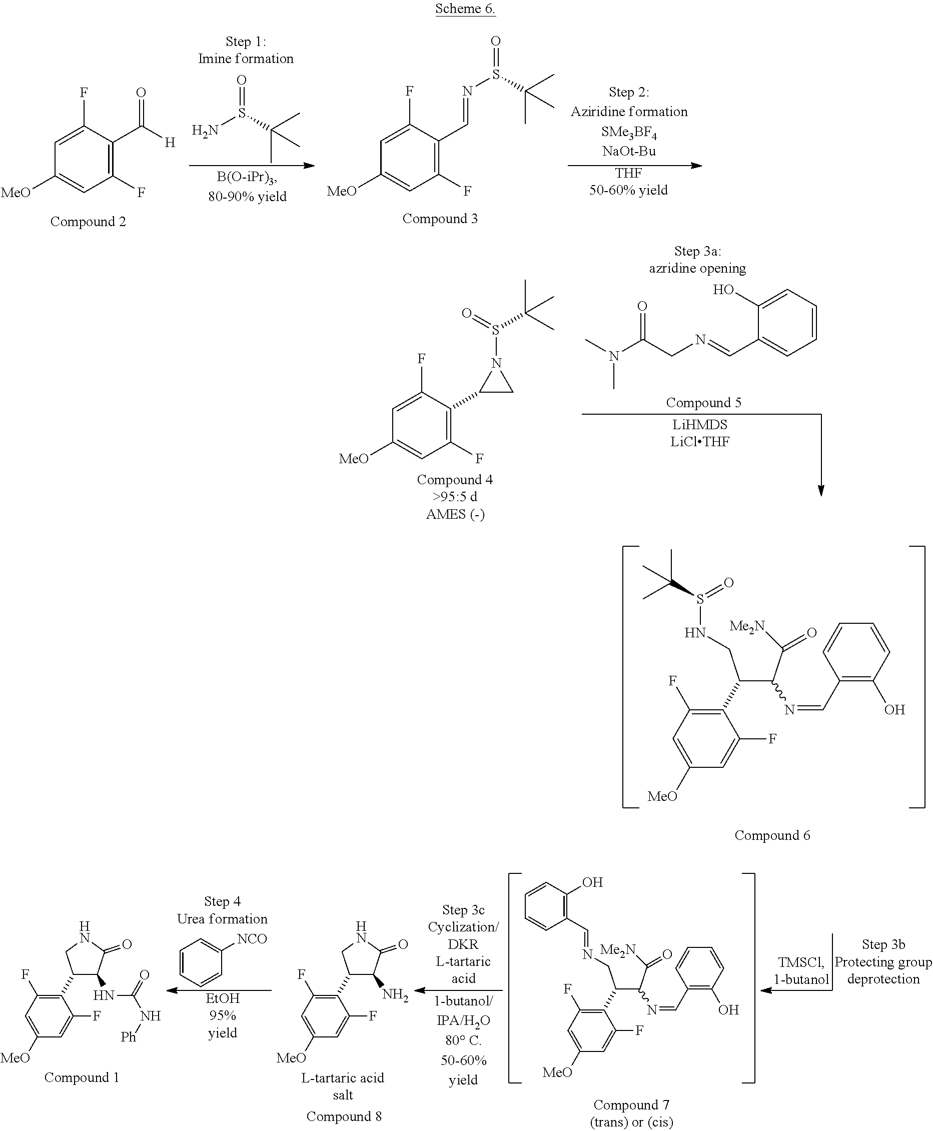

### Example 1

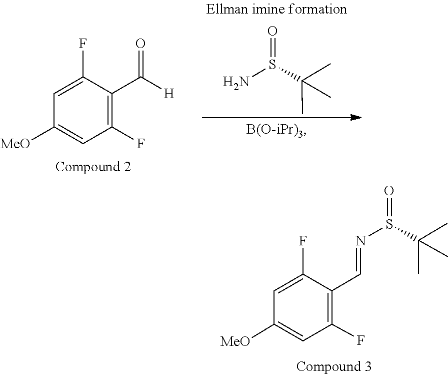

To a 20-L reactor was added Compound 2 (1 kg, 1 eq). Trisopropylborate (4 L) was added followed by and (R)-(±)-2-methylpropane-2-sulfinamide (810 g, 1.15 eq). The resulting slurry was heated to 65-70° C. for 18 h during which time the reaction mixture became homogeneous. The reaction mixture was cooled to 0° C. over 18 h resulting in a thick shiny. The shiny was held at 0° C. for 1 h before filtering off the solids. The solids were washed with heptane/MTBE (4 L) and the solids were dried at 55° C. resulting in Compound 3 (1.42 Kg, 89% yield) as an off-white crystalline solid. ¹H NMR (600 MHz, C₆D₆): δ9.08 (s, 1H), 6.08 (d, J=10.3 Hz, 2H), 2.96 (s, 3H), 1.14 (s, 9H). ¹³C NMR (150 MHz, C₆D₆): δ 164.4 (t, J=14.6 Hz), 163.8 (dd, J=257.7, 9.4 Hz), 153.0, 106.5 (t, J=12.6 Hz), 99.1 (d, J=24.8 Hz), 57.6, 55.8, 22.8. HRMS (ESI) Calcd for [C₁₂H₁₅F₂NO₂S+H]⁺ 276.0864, Found 276.0867 (0.9 ppm error).

### Example 2

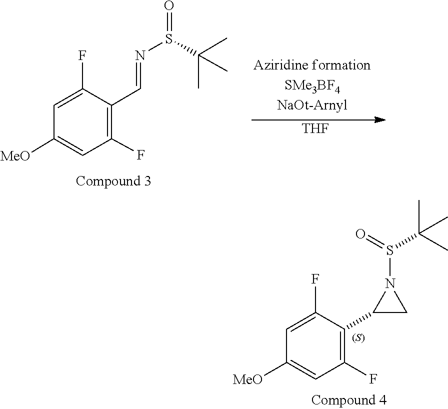

To a 20-L reactor was added Compound 3 (1 Kg, 1 eq) Me₃SBF₄ (715 g, 1.2 eq), and THF (15 L, 15 V). The resulting slurry was cooled to 15° C. and a solution of Na tert-pentoxide (1.4 M in THF, 3.1 L, 1.2 eq) was added over no less than 2 h while maintaining an internal reaction temp between 18-22° C. The reaction mixture was quenched with 10% aq. NH₄OAc (5 L, 5 ml/g). n-Octane (5 L, 5 ml/g) was added to the mixture to facilitate extraction. The layers were split and the organic stream was washed with 13% brine (3×5 L, 5 ml/g). The rich organic stream was concentrated under reduced pressure to ca. 3 total volumes, the solvent swapped to n-octane under constant volume conditions (full vacuum, 70 C). The batch is cooled to 30° C. and seeds are added (10 g, 1 wt %). The resulting slurry is aged at 30° C. for 2 h then cooled to 15° C. over 18 h. The resulting solids are filtered and washed with pre-cooled (−5 to 0 C) n-octane (1 L, 1 vol). The resulting solids are dried under vacuum at 30-35 C to yield Compound 4 (590 g, 52.6?% yield). ¹H NMR (600 MHz, C₆D₆): δ 6.24 (d, J=10.6 Hz, 2H), 4.01 (br s, 1H), 3.15 (s, 3H), 2.57 (br s, 1H), 2.11 (br d, J=7.3 Hz, 1H), 1.07 (s, 9H). ¹³C NMR (150 MHz, C₆D₆): δ 163.6 (dd, J=248.7, 11.3 Hz), 161.1 (t, J=14.1 Hz), 105.3 (t, J=14.8 Hz), 99.0 (br d, J=24.3 Hz), 56.9, 55.7, 28.3, 24.0, 22.9. HRMS (ESI) Calcd for [C₁₃H₁₇F₂NO₂S+H]⁺ 290.1021, Found 290.1024 (1.1 ppm error). MP=66-67° C. Compound 4 was tested and was AMES (−).

### Example 3

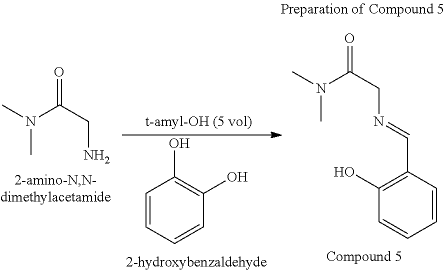

To a 10-L reactor was added 2-amino-N,N-dimethylacetamide (1.0 Kg, 1 eq) and t-Amyl-OH (5 L, 5 vol). To this mixture was added 2-hydroxybenzaldehyde (1.25 eq) at 20° C. over 30 min period. Upon completion of the addition, the reaction mixture was heated to 40 C for 12 h. The resulting slurry was cooled 0-5° C. and age for no less than 2 h. The solids were filtered and washed solids with cold t-AmylOH (4 L, 4 vol), followed by MTBE (2 L, 2 vol). The resulting yellow crystalline solids were dried under vacuum at Solids are dried at 50-60° C. for 12 h to afford Compound 5 (1.72 Kg, 89% yield). Compound 5. ¹H NMR (600 MHz, acetone-d₆): δ 13.29 (br s, 1H), 8.50 (s, 1H), 7.40 (dd, J=7.8, 1.7 Hz, 1H), 7.33 (m, 1H), 6.90, (overlap, 1H), 6.89 (overlap, 1H), 4.52 (s, 2H), 3.11 (s, 3H), 2.91 (s, 3H). ¹³C NMR (150 MHz, acetone-d₆): δ 169.2, 168.7, 162.1, 133.2, 132.7, 120.0, 119.4, 117.5, 60.4, 36.9, 35.4. HRMS (ESI) Calcd for [C₁₁H₁₄N₂O₂+H]⁺207.1128, Found 207.1129 (0.3 ppm error).

### Example 4

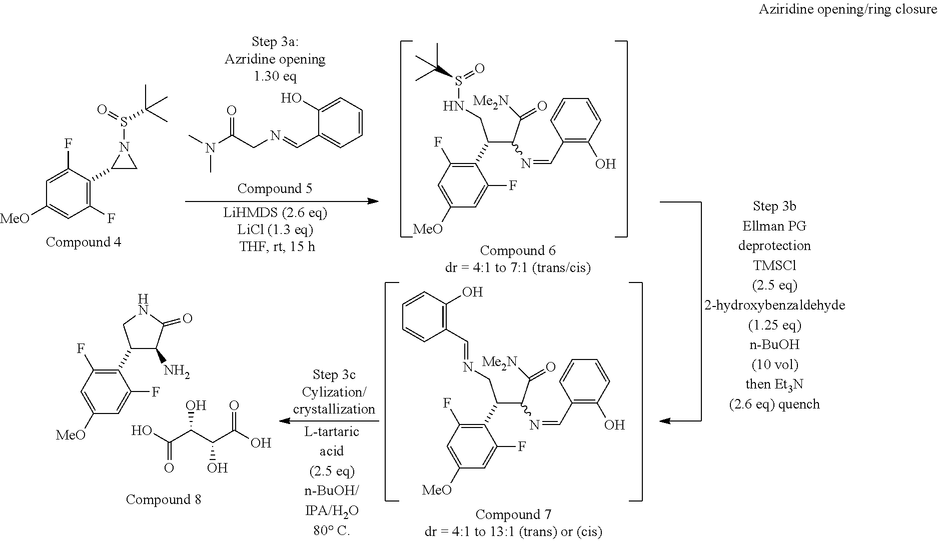

To a 20-L reactor was added THF (10 L, 10 L/kg) and LiCl (190 g, 1.30 eq). The resulting slurry was stirred at 20° C. for 30 min to dissolve LiCl. To the reaction mixture was added 927 g BMT-Compound 5 (927 g, 1.30 eq) and the resulting mixture was agitated for 30 min before being cooled to 10-15° C. LiHMDS (8.81 L, 1.0 M in THF, 2.55 eq) was added at such a rate that the internal temperature did not exceed 25° C. The reaction mixture was warmed to 20-25° C. and agitated for 30 min before Compound 4 (1 kg, 1.0 eq) was added as a solid and agitation of the reaction is continued at this temperature for an additional 16 h. The reaction mixture was quenched with 20 wt % aq. NH₄OAc (10 L, 10 Vol) and the resulting layers are split. The organic stream was washed with 20 wt % NH₄OAc (10 L, 10 vol) and the resulting layers are split. The organic layer is concentrated under reduced pressure to a final volume of ca. 10 vol. A constant volume distillation is conducted to swap THF solvent for 1-butanol. The reaction mixture, now a thick slurry of Compound 6 is cooled to 15° C. and salicylaldehyde (437 mL, 1.20 eq) was added followed by TMSCl (1.1 L, 2.5 eq) at such a rate that the internal temperature remained <25° C. During this time the reaction mixture becomes homogenous and obtains a red color. The reaction mixture is warmed to 20-25° C. and held at this temperature for 1 h before cooling to 15° C. To this mixture was added TEA (1.25 L, 2.6 eq) resulting in a yellow slurry of Compound 7. To this mixture was added THF (10 L, 10 L/kg) and then the reaction was quenched with 13 wt % aq. NaCl (5 L, 5 vol). The layers are split and the organic stream is washed with H₂O (5 L, 5 vol). The layers are split and organic stream is concentrated under reduced pressure to ca. 8 Vol total. (20-50 mbar, max jacket set to 85° C.). The resulting slurry of Compound 7 was cooled to 15-25° C. and IPA (8 L, 8 L/Kg) was added and the mixture was heated 50° C. In a separate vessel a solution was prepared of L-tartaric acid (1.297 Kg, 2.5 eq) in (4 L, 4 vol). This aqueous solution of L-tartaric acid was added to the above reaction mixture at 50° C. over a period of 30 min. The resulting mixture was heated to 75-80° C. for 16 h before being cooled to 45° C. over 2 h period and then aged for 6 h to result in a thick slurry of Compound 8. The slurry was cooled to 5° C. over 12 h and aged for 2 h. The solids are filtered and washed IPA/H₂O (80:20, 6 L, 6 vol) and then with IPA (4 L, 4 vol) to yield Compound 8 (875 g, 70% yield) as an L-tartaric acid salt.

Compound 6: ¹H NMR (600 MHz, CDCl₃): δ 12.39 (br s, 1H), 8.23 (s, 1H), 7.23 (ddd, 8.1, 7.4, 1.6 Hz, 1H), 7.12 (dd, J=7.7, 1.5 Hz, 1H), 6.82 (d, J=8.2 Hz, 1H), 6.79 (td, J=7.5, 0.9 Hz, 1H), 6.34 (d, J=10.7 Hz, 2H), 4.91 (d, J=10.5 Hz, 1H), 4.12 (td, J=9.6, 4.8 Hz, 1H), 3.67 (s, 3H), 3.67 (m, 1H), 3.56 (dt, J=12.8, 5.0 Hz, 1H), 3.38 (dt, J=12.8, 9.0 Hz, 1H), 3.11 (s, 3H), 3.00 (s, 3H), 1.03 (s, 9H). ¹³C NMR (150 MHz, CDCl₃): δ 169.2, 166.7, 162.3 (dd, J=244.5, 12.1 Hz), 160.9, 160.2 (t, J=14.2 Hz), 132.9, 132.1, 118.8, 118.6, 106.9 (t, J=18.5 Hz), 98.2 (d, J=27.4 Hz), 66.9, 55.82, 55.77, 47.2, 40.4, 37.3, 36.4, 22.6. HRMS (ESI) Calcd for [C₂₄H₃₁F₂N₃O₄S+H]⁺ 496.2076, Found 496.2085 (1.8 ppm error).

Compound 7: ¹H NMR (600 MHz, CDCl₃): δ 12.95 (br s, 1H), 12.53 (br s, 1H), 8.34 (s, 1H), 8.31 (s, 1H), 7.30 (ddd, J=9.4, 7.4, 1.7 Hz, 1H), 7.29 (ddd, J=9.0, 7.4, 1.7 Hz, 1H), 7.22 (dd, J=7.8, 1.6 Hz, 1H), 7.18 (dd, J=7.6, 1.6 Hz, 1H), 6.94 (d, J=8.3 Hz, 1H), 6.89 (d, J=8.3 Hz, 1H), 6.87 (td, J=7.5, 0.9 Hz, 1H), 6.85 (td, J=7.4, 1.0 Hz, 1H), 6.40 (d, J=10.9 Hz, 2H), 5.11 (d, J=10.6 Hz, 1H), 4.43 (ddd, J=10.4, 8.3, 5.3 Hz, 1H), 4.03 (dd, J=12.4, 8.3 Hz, 1H), 3.98 (dd, J=12.4, 5.3 Hz, 1H), 3.70 (s, 3H), 3.20 (s, 3H), 3.02 (s, 3H). ¹³C NMR (150 MHz, CDCl₃): δ 169.1, 166.8, 166.3, 162.2 (dd, J=245.2, 12.1 Hz), 161.1, 160.9, 160.2 (J=14.5 Hz), 133.0, 132.5, 132.2, 131.6, 118.8, 118.72, 118.67, 117.2, 117.1, 106.7 (t, J=18.5 Hz), 98.4 (d, J=27.6 Hz), 67.9, 60.6, 55.8, 39.8, 37.3, 36.4. HRMS (PSI) Calcd for [C₂₇H₂₇F₂N₃O₄+H]⁺ 496.2042, Found 496.2049 (1.4 ppm error).

Compound 8: ¹H NMR (600 MHz, DMSO-d₆): δ 8.31 (s, 1H), 6.76 (d, J=10.7 Hz, 2H), 4.01 (br s, 2H), 3.85 (d, J=10.6 Hz, 1H), 3.77 (s, 3H), 3.68 (m, 1H), 3.49 (t, J=9.2 Hz, 1H), 3.27 (t, J=9.5 Hz, 1H). ¹³C NMR (150 MHz, DMSO-d₆): δ 174.0, 172.9, 161.8 (dd, J=245.0, 11.8 Hz), 160.1 (t, J=14.6 Hz), 106.0 (t, J=17.9 Hz), 98.7 (d, J=27.2 Hz), 71.7, 56.1, 54.7, 43.1, 36.2. HRMS (ESI) Calcd for [C₁₁H₁₂F₂N₂O₂+H]⁺ 243.0940, Found 243.0939 (0.4 ppm error).

### Example 5

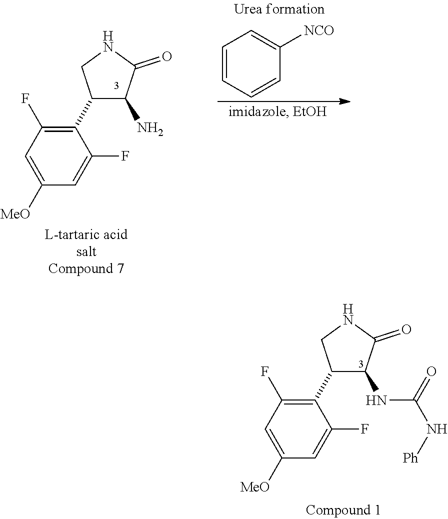

To a reactor was added EtOH (200 proof, 10 vol, 10 L) and imidazole (0.61 Kg, 3.5 eq). To the resulting mixture was added Compound 8 (1 Kg, 1 eq) to give a slurry. To this slurry was added Phenylisocyanate (0.33 kg, 1.1 eq.) over no less than 30 minutes to yield Compound 1 after a “work up.” Compound 1 ¹H NMR (600 MHz, DMSO-d₆): δ 8.61 (s, 1H), 8.06 (s, 1H), 7.33 (br d, J=8.2 Hz, 2H), 7.19 (br t, J=7.8 Hz, 2H), 6.88 (br t, 7.3 Hz, 1H), 6.74 (d, J=10.9 Hz, 2H), 6.46 (d, J=8.4 Hz, 1H), 4.59 (dd, J=10.9, 8.4 Hz, 1H), 3.80 (m, 1H), 3.76 (s, 3H), 3.46 (br t, J=9.1 Hz, 1H), 3.32 (br t, J=9.6 Hz, 1H). ¹³C NMR (150 MHz, DMSO-d₆): δ 173.5, 161.8 (dd, J=244.0, 11.9 Hz), 159.7 (t, J=14.6 Hz), 154.9, 140.1, 128.6, 121.2, 117.7, 106.9 (t, J=17.6 Hz), 98.6 (d, J=28.3 Hz), 56.0, 54.6, 42.4, 36.4. HRMS (ESI) Calcd for [C₁₈H₁₇F₂N₃O₃+H]⁺ 362.1311, Found 362.1312 (0.3 ppm error).

### Example 6

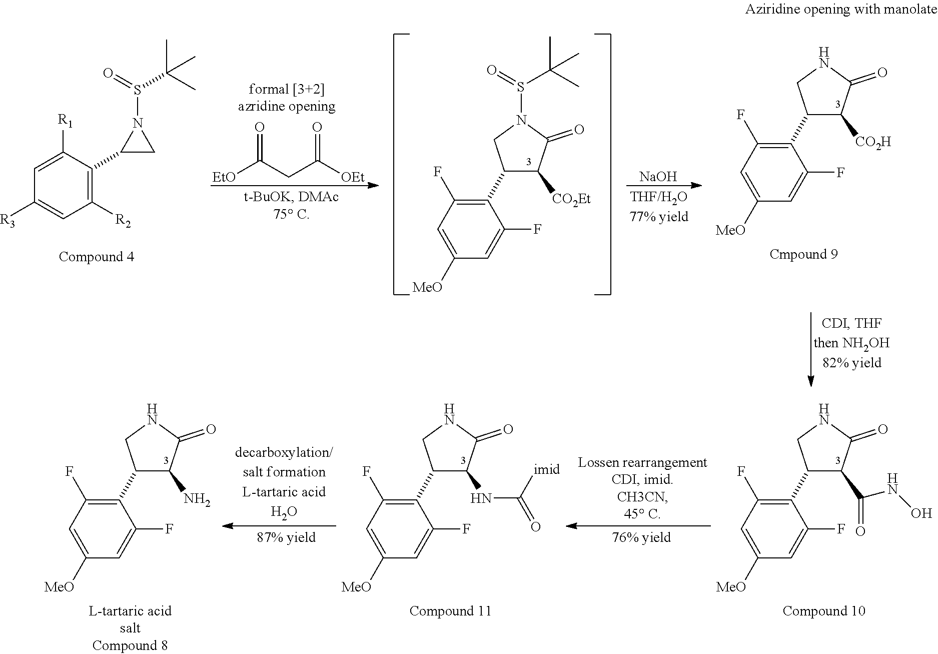

In the process, Compound 4 was reacted with diethylmalonate. After treatment with NaOH, the resulting Compound 9 could be isolated in 77% yield. The C-3 carboxylic acid was transformed to the desired C-3 amino group via Curtius reaction or by Lossen rearrangement, in both cases converging on the same Compound 11 imidazole adduct. Compound 8 was accessed by treatment with tartaric acid and water (87% yield).

### Example 7

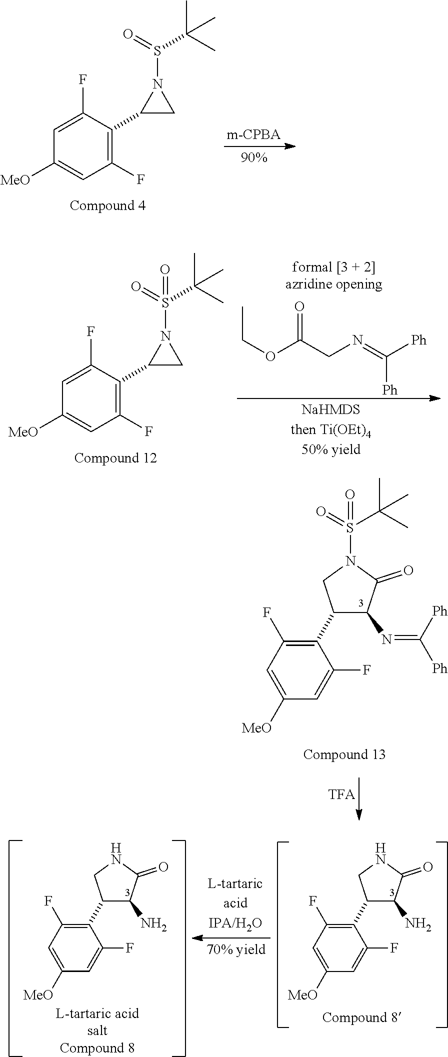

Compound 4 was oxidized by m-CPBA to a more reactive Bus-aziridine, Compound 12, which was then reacted with benzophenone glycine imine ethyl ester (50% yield). Removal of the Bus-group was conducted with anhydrous TFA and then telescoped into the tartaric acid salt formation (70% yield) to afford Compound 8.

### Example 8

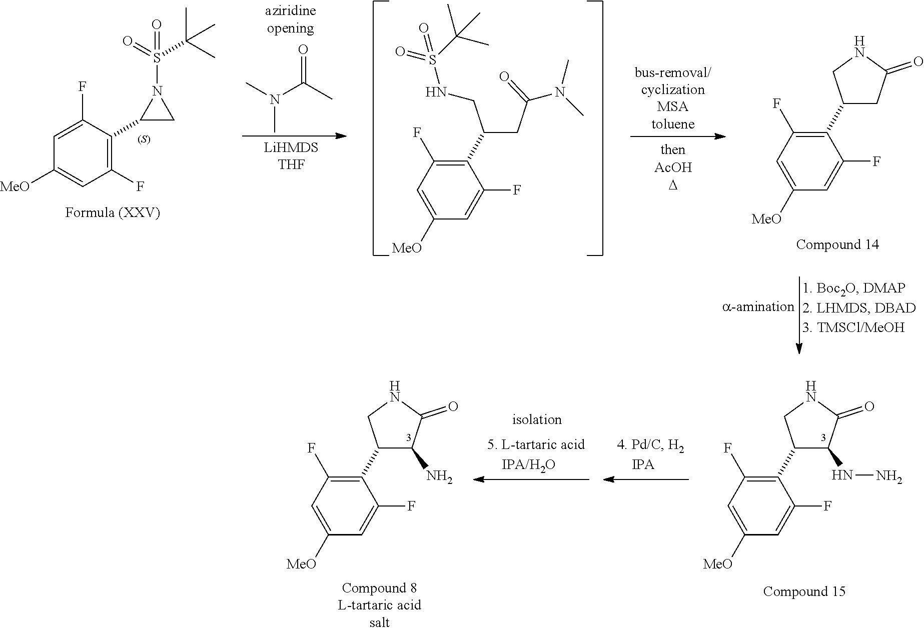

In an alternative route, Compound 13 was opened with DMAc enolate. Cyclization was successful by first removing the Bus group with MSA/toluene at reflux and then treatment with AcOH at reflux. Installation of the C-3 Amino group was carried out in a 3 step process starting with N-Boc protection, alpha amination with DBAD, and then treatment with TMSCl produced the resulting C-3 hydrazine intermediate. Scission of the N—N bond was carried out with Pd/C and then Compound 8 was generated after treatment with L-tartaric acid.

1. A process for the preparation of a compound of Formula (I) 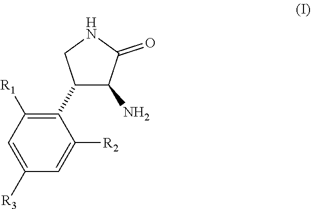
or a salt thereof, wherein each of R₁ and R₂ is halogen and R₃ is C₁₋₄ alkoxy, comprising the steps of
(1) condensing a sulfonamide chiral auxiliary with a substituted phenyl aldehyde in a solvent to provide an imine product;
(2) reacting the resulted imine product with a sulfonium-ylide to afford an aziridine electrophile;
(3) reacting the aziridine electrophile with an enolate nucleophile to afford the compound of Formula (I).

2. The process of claim 1, wherein the phenyl aldehyde is a compound of Formula (II): 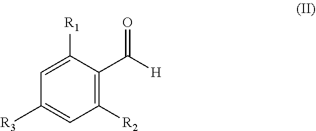
wherein each of R₁ and R₂ is halogen and R₃ is C₁₋₄ alkoxy.

3. The process of claim 1, wherein the sulfonamide chiral auxiliary is

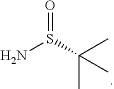

4. The process of claim 1, wherein the imine product is a compound of Formula (III): 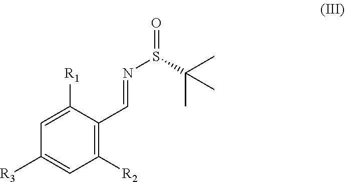
wherein each of R₁ and R₂ is halogen and R₃ is C₁₋₄ alkoxy.

5. The process of claim 1, wherein the solvent is B(i-PrO)₃.

6. The process of claim 1, wherein the sulfonium-ylide is generated from a suitable salt and a suitable base.

7. The process of claim 6, wherein the salt is selected from the group consisting of SMe₃BF₄, SMe₃Cl, SMe₃Br, SMe₃I, and SMe₃PF₆.

8. The process of claim 7, wherein the salt is SMe₃BF₄.

9. The process of claim 6, wherein the base is selected from the group consisting of sodium hydroxide, potassium hydroxide, potassium t-butoxide, sodium t-butoxide, sodium methoxide, potassium methoxide, sodium ethoxide, potassium ethoxide, sodium tert-pentoxide (NaOt-Amyl), potassium tert-pentoxide sodium isopropoxide, and potassium isopropoxide.

10. The process of claim 9, wherein the base is NaOt-Amyl.

11. The process of claim 6, wherein the reaction is conducted at a temperature in the range of about −10° C. to 20° C.

12. The process of claim 1, wherein the aziridine electrophile is a compound of Formula (IV): 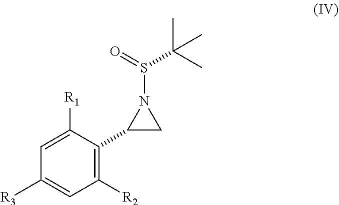
wherein each of R₁ and R₂ is halogen and R₃ is C₁₋₄ alkoxy.

13. The process of claim 1, wherein each of R₁ and R₂ is F; and R₃ is methoxy.

14. The process of claim 1, wherein the enolate nucleophile is a glycine imine derivative of Formula (V): 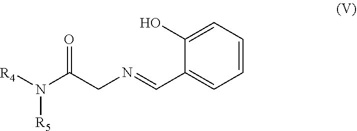
wherein
R₄ and R₅ are independently selected from the group consisting of H, C₁₋₃ alkyl, C₃₋₆ cycloalkyl, phenyl, and 5- to 6-membered heterocycle containing carbon atoms and 1-4 heteroatoms selected from the group consisting of N, O, and S.

15. The process of claim 14, wherein the compound of Formula (V) is reacted with a base in an organic solvent in the presence of LiCl to form a lithium dianion.

16. The process of claim 1, wherein an intermediate generated from Step (3) is a compound of Formula (VI): 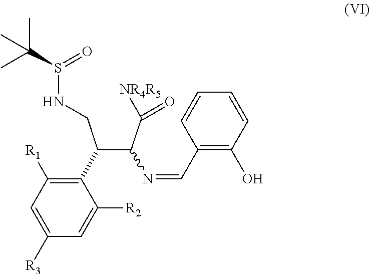
wherein:
each of R₁ and R₂ is halogen;
R₃ is C₁₋₄ alkoxy; and
R₄ and R₅ are independently selected from the group consisting of H and C₁₋₃ alkyl.

17. The process of claim 1, wherein Step (3) further comprises the steps of
3(a) replacing the sulfonamide auxiliary protecting group of the compound of Formula (VI) with a Schiff base protecting group; and
3(b) removing the Schiff base protecting group and cyclizing the compound.

18. The process of claim 17, wherein in Step 3(a), the compound of Formula (VI) is reacted with an acid and in the presence of 2-hydroxybenzaldehyle to afford a compound of Formula (VII): 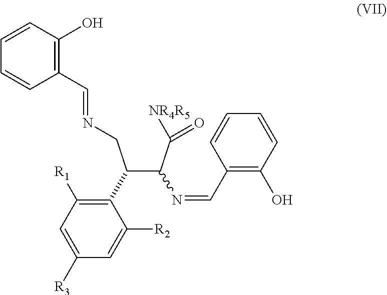
wherein:
each of R₁ and R₂ is halogen;
R₃ is C₁₋₄ alkoxy; and
R₄ and R₅ are independently selected from the group consisting of H and C₁₋₃ alkyl.

19. The process of claim 17, wherein in Step 3(b), the compound of Formula (VII) is treated with a chiral acid in a mixture of water and an alcohol to afford a compound of Formula (I).

20. The process of claim 19, wherein the chiral acid is tartaric acid.

21. The process of claim 19, wherein the alcohol is selected from the group consisting of methanol, ethanol, propanol, isopropanol, and butanol.

22. The process of claim 21, wherein the alcohol is isopropanol/1-butanol.

23. The process of claim 19, wherein the reaction is conducted at a temperature in the range of about 70° C. to 80° C.

24. A process for the preparation of a compound of Formula (I): 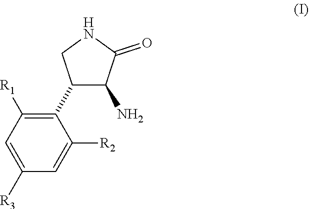
wherein each of R₁ and R₂ is halogen and R₃ is C₁₋₄ alkoxy, comprising the steps of
(1) reacting the compound of Formula (IV): 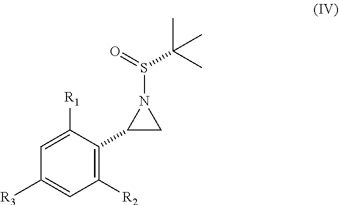
with a benzophenone glycine imine ester;
(2) treating the resultant product with a chiral acid in an alcohol to afford a compound of Formula (I).

25. A process for the preparation of a compound of Formula (I): 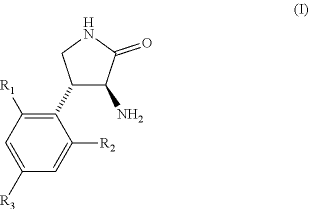
wherein each of R₁ and R₂ is halogen and R₃ is C₁₋₄ alkoxy, comprising the steps of (1) reacting the compound of Formula (IV): 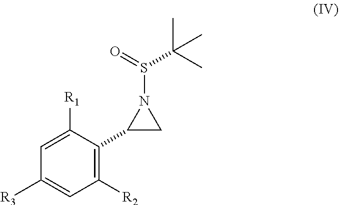
with a malonate derivative;
(2) treating the resultant product with base to afford a compound of Formula (VIII): 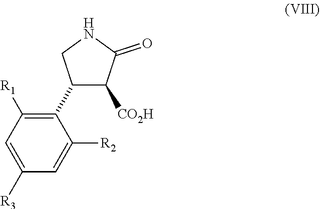
(3) converting the compound of Formula (VIII) into a hydroxamic acid of Formula (IX): 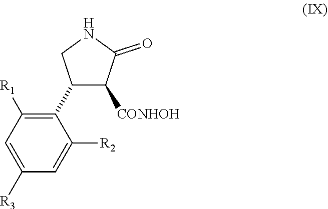
(4) Converting the hydroxoamic acid by Lossen rearrangement to afford a compound of Formula (X): 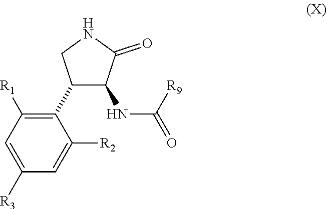
wherein R₉ is 5- to 6-membered heterocycle containing carbon atoms and 1-4 heteroatoms selected from the group consisting of N, O, and S;
(5) treating the resultant product with tartaric acid to afford a compound of Formula (I).

26. The process of claim 25, wherein the malonate is diethylmalonate.

27. A process for the preparation of a compound of Formula (I): 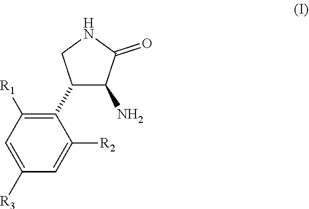
wherein each of R₁ and R₂ is halogen and R₃ is C₁₋₄ alkoxy, comprising the steps of
(1) oxidizing the compound of Formula (IV): 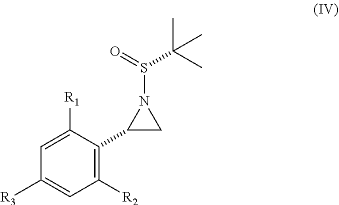
with an oxidizing agent to afford a compound of Formula (XI): 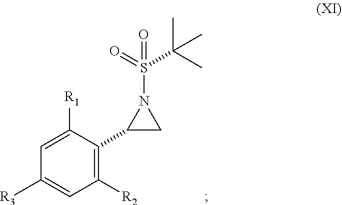
(2) reacting the compound of Formula (XI) with a glycine imine ester; and
(3) treating the resultant product with a chiral acid in an alcohol to afford a compound of Formula (I).

28. A process for the preparation of a compound of Formula (I): 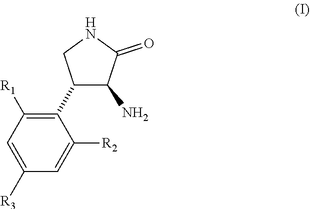
wherein each of R₁ and R₂ is halogen and R₃ is C₁₋₄ alkoxy, comprising the steps of
(1) reacting the compound of Formula (XI): 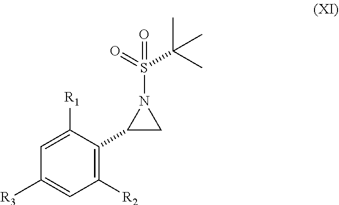
with a substituted acetamide and cyclizing the compound to afford a compound of Formula (XII): 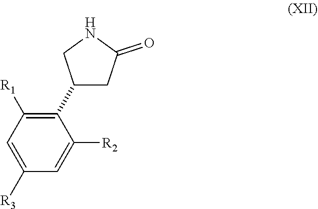
(2) aminating the compound of Formula (XII) with DBAD to afford a compound of Formula (XIII): 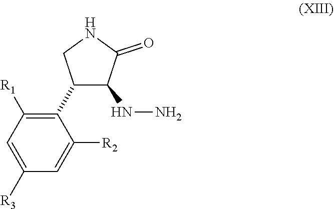
(3) reducing the compound of Formula (XIII) to afford a compound of Formula (I).

29. A process for the preparation of Compound (XIV): 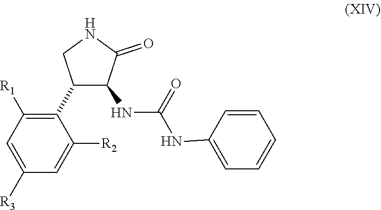
wherein each of R₁ and R₂ is halogen and R₃ is C₁₋₄ alkoxy: comprising the steps of
(1) condensing a sulfonamide chiral auxiliary with a substituted phenyl aldehyde in a solvent to provide an imine product;
(2) reacting the resulted imine product with a sulfonium-ylide to afford an aziridine electrophile;
(3) reacting the aziridine electrophile with an enolate nucleophile to afford the compound of Formula (I); 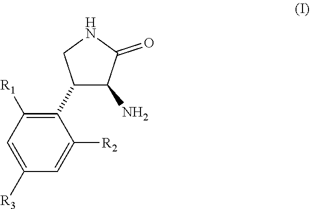
wherein R₁, R₂, and R₃ are as defined above;
(4) coupling the compound of Formula (I) with phenylisocyanate in the presence of an alcoholic solvent and a base to afford the compound of Formula (XIV).

30. The process of claim 29, wherein each of R₁ and R₂ is F, and R₃ is methoxy.

31. The process of claim 30, wherein the base is imidazole.

32. A compound of Formula (XV): 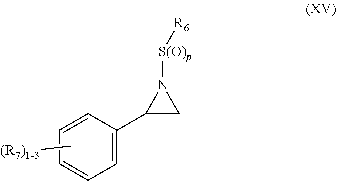
wherein
R₆ is C₁₋₆alkyl;
R₇ is selected from the group consisting of halogen, OH, C₁₋₄alkyl, C₂₋₄ alkenyl, C₁₋₄alkoxy, C₁₋₄alkylthio, C₁₋₄haloalkyl, —CH₂OH, —OCH₂F, —OCHF₂, —OCF₃, CN, —NH₂, —NH(C₁₋₄ alkyl), —N(C₁₋₄ alkyl)₂, —CO₂H, —CH₂CO₂H, —CO₂(C₁₋₄ alkyl), —CO(C₁₋₄ alkyl), —CH₂NH₂, —CONH₂, —CONH(C₁₋₄ alkyl), and —CON(C₁₋₄ alkyl)₂; and
p is an integer of 1 or 2.

33. The compound of claim 32 having the structure:

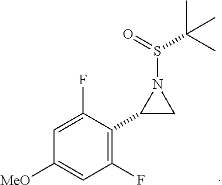

34. The compound of claim 32 having the structure:

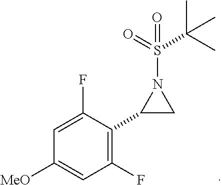

35. A compound of Formula (V): 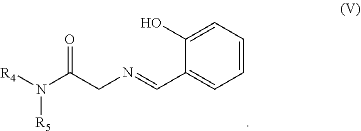
wherein R₄ and R₅ are independently selected from the group consisting of H, C₁₋₄alkyl, C₃₋₆ cycloalkyl, phenyl, and 5- to 6-membered heterocycle containing carbon atoms and 1-4 heteroatoms selected from the group consisting of N, O, and S.

36. The compound of claim 35 having the structure:

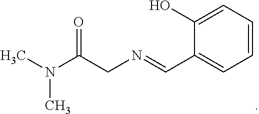

37. A compound of Formula (XVII): 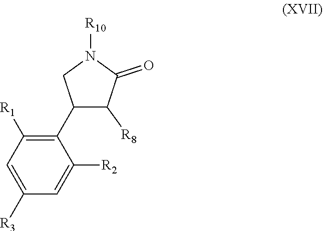
wherein
each of R₁ and R₂ is halogen;
R₃ is C₁₋₄ alkoxy;
R₈ is selected from the group consisting of —CO₂R₉, —CONH—OH, —NHCOR₉, —N═C(R₉)₂, —N(R₉)₂, and —NH—NH₂;
R₉ is selected from the group consisting of H, C₁₋₄alkyl, C₃₋₆ cycloalkyl, aryl, and 5- to 6-membered heterocycle containing carbon atoms and 1-4 heteroatoms selected from the group consisting of N, O, and S; and
R₁₀ is selected from the group consisting of H, S(O)C₁₋₆ alkyl, and S(O)₂C₁₋₆alkyl.

38. The compound of claim 37, wherein
each of R₁ and R₂ is F;
R₃ is methoxy;
R₈ is selected from the group consisting of —CO₂H, —CONH—OH, —NHCO-imidazole, —N═C(Ph)₂, —NH₂, and —NH—NH₂; and
R₁₀ is H.

39. The compound of claim 37, wherein
each of R₁ and R₂ is F;
R₃ is methoxy;
R₈ is selected from the group consisting of —CO₂H—CONH—OH, —NHCO-imidazole, —N═C(Ph)₂, —NH₂, and —NH—NH₂; and
R₁₀ is selected from the group consisting of S(O)C₁₋₆ alkyl and S(O)₂C₁₋₆alkyl.

40. A compound of Formula (XVIII): 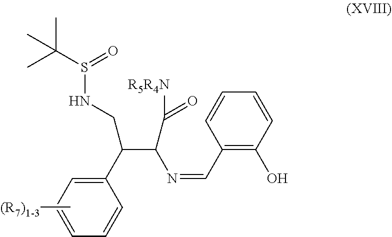
wherein R₇ is selected from the group consisting of halogen, OH, C₁₋₄alkyl, C₂₋₄ alkenyl, C₁₋₄alkoxy, C₁₋₄alkylthio, C₁₋₄haloalkyl, —CH₂OH, —OCH₂F, —OCHF₂, —OCF₃, CN, —NH₂, —NH(C₁₋₄ alkyl), —N(C₁₋₄ alkyl)₂, —CO₂H, —CH₂CO₂H, —CO₂(C₁₋₄ alkyl), —CO(C₁₋₄ alkyl), —CH₂NH₂, —CONH₂, —CONH(C₁₋₄ alkyl), and —CON(C₁₋₄ alkyl)₂; and
R₄ and R₅ are independently selected from the group consisting of H and C₁₋₃ alkyl.

41. A compound of Formula (XIX): 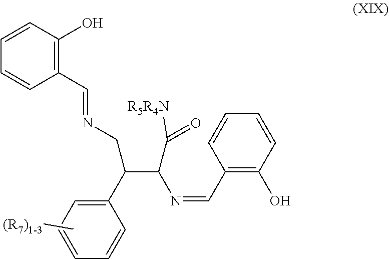
wherein R₇ is selected from the group consisting of halogen, OH, C₁₋₄alkyl, C₂₋₄ alkenyl, C₁₋₄alkoxy, C₁₋₄alkylthio, C₁₋₄haloalkyl, —CH₂OH, —OCH₂F, —OCHF₂, —OCF₃, CN, —NH₂, —NH(C₁₋₄ alkyl), —N(C₁₋₄ alkyl)₂, —CO₂H, —CH₂CO₂H, —CO₂(C₁₋₄ alkyl), —CO(C₁₋₄ alkyl), —CH₂NH₂, —CONH₂, —CONH(C₁₋₄ alkyl), and —CON(C₁₋₄ alkyl)₂; and
R₄ and R₅ are independently selected from the group consisting of H and C₁₋₃ alkyl.
